(19) United States
(12) Patent Application Publication
(10) Pub. No.: US 2004/0077676 A1
(43) Pub. Date: Apr. 22, 2004

(54) Neurotrophic tacrolimus analogs

(76) Inventors: Nobuya Matsuoka, Osaka-shi (JP); Takayuki Yamaji, Osaka-shi (JP); Bruce Gold, West Linn, OR (US)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,361

(22) PCT Filed: Dec. 31, 2001
(86) PCT No.: PCT/US01/50419

(51) Int. Cl. A61K 31/4745

(52) U.S. Cl. 514/291

(57) ABSTRACT

Tacrolimus derivatives having high levels of neurotrophic activity and low levels of immunosuppressive activity. These compounds are useful as neurotrophic agents, particularly, for preventing or treating neuronal injury/dysfunction.

### BACKGROUND OF THE INVENTION

1. Field of the Invention

This invention relates to a tacrolimus derivative having a high level of neurotrophic activity and a low level of immunosuppressive activity.

2. Discussion of the Background

Certain macrolide compounds, e.g. tacrolimus and related compounds, are known to help prevent or treat cerebral ischemia (W094/14443). Particular pipecolic acid derivatives that have affinity for FKBP-type immunophilins, such as tacrolimus, are known to stimulate growth of damaged peripheral nerves or promote neuronal regeneration (WO96/40140). Certain non-immunosuppressive compounds, i.e., geldanamycin and its analogs, are shown to disrupt the steroid receptor complex and promote nerve growth (WO99/21552).

### SUMMARY OF THE INVENTION

The present inventors have found that a particular tacrolimus analogue, i.e. Compound (I), mentioned below, has an excellent neurotrophic activity but, unlike tacrolimus, has little or no immunosuppressive activity. As shown below, Compound (I) exerts superior levels of neurotropic activity compared to tacrolimus, for instance, as measured by its ability to increase neurite length. Similarly, the administration of Compound (I) is shown to induce axonal regeneration and speed recovery from nerve crush or spinal cord injuries. Moreover, Compound (I) exerts these advantageous neurotropic effects with little or no immunosuppressive activity compared to tacrolimus.

Accordingly, the present invention provides new uses for Compound (I) as a superior neurotrophic agent, as well as a neurotropic agent with little or no immunosuppressive activity.

Further, the invention provides a neurotrophic agent or composition that comprises Compound (I).

Still further, this invention provides a method for preventing or treating neuronal injury/dysfunction that comprises administering Compound (I) to a mammal.

### DETAILED DESCRIPTION OF THE INVENTION

Unexpectedly, the present inventors have discovered that Compound (I) is useful for ameliorating, preventing, or treating neurological injury or dysfunction caused by damage or injury to, deterioration of, or disease of the nervous system, while advantageously having little or no immunosuppressive effect.

Compound (I) is useful for treating damage, deterioration or dysfunction caused by physical injury, nutritional disorders, ischemia, degenerative diseases, malignant diseases, infectious diseases, and by drug interactions, toxins or poisons. For instance, Compound (I) is useful for treating neurological damage or dysfunction caused by neurosurgery, peripheral nerve injury, burns, encephalomyelitis, HIV, herpes, cancer, radiation treatment, drug interaction, folic acid or Vitamin B-12 deficiency, and by exposure to neurotoxins or chemicals such us lead.

Accordingly, Compound (I) is useful for preventing or treating neuronal injury and dysfunction, such as polymyositis (multiple myositis), Guillan-Barre syndrome, Meniere's disease, polyneuritis (multiple neuritis), mononeuritis (solitary neuritis), Alzheimer's disease, Parkinson's disease, amyotrophic lateral sclerosis (ALS), Huntington's disease, radiculopathy, neuropathy (such as diabetic neuropathy, chemotherapy-induced neuropathy, etc.), spinal cord injury, senile dementia, vascular dementia, multiple sclerosis, physical palsy, etc.

Compound (I), the tacrolimus analog used in the present invention, has the following chemical formula:

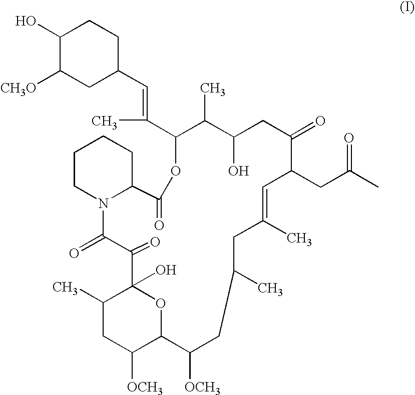

This compound may be produced as described by U.S. Pat. No. 5,376,663, Example 29. With respect to Compound (I) used in the present invention, it is to be understood that there may be conformers and one or more stereoisomers, such as optical and geometrical isomers due to asymmetric carbon atom(s) or double bond(s), and such conformers and isomers are also included within the scope of the compound in the present invention.

Compound (I) may also be in the form of a pharmaceutically acceptable salt, derivative, solvate or pro-drug, all of which are included within the scope of the present invention. The solvate preferably includes a hydrate and an ethanolate.

A preferable form of Compound (I) is the following one:

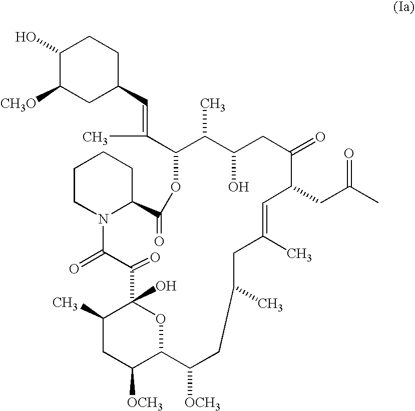

Compound (I) in the present invention may be administered as a pure compound or as a mixture with another compound or other ingredients, preferably, in a pharmaceutical vehicle or carrier. When Compound (I) is used in the form of a pharmaceutical preparation or composition it may be admixed with an organic or inorganic carrier, vehicle or excipient suitable for external (topical), oral, enteral, subcutaneous, intravenous, intramuscular, or parenteral applications. For example, it may be present in solid, semisolid or liquid composition, which contains Compound (I) as an active ingredient and one or more carriers, vehicles or excipients. Typical carriers, vehicles or excipients include, but are not limited to conventional pharmaceutical carriers, medicinal or pharmaceutical agents, buffers, dispersants, emulsifying agents and adjuvants.

Compound (I) may also be compounded with the usual non-toxic, pharmaceutically acceptable carriers for tablets, pellets, capsules, eye drops, suppositories, solutions (saline, for example), emulsions, suspensions (olive oil, for example), ointments, aerosol sprays, creams, skin plasters, patches and any other form suitable for use.

Suitable carriers include water, aqueous saline and dextrose solutions, oils, including animal, vegetable and synthetic oils, and petroleum products. Other useful carriers include glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semisolid, or liquid form. In addition auxiliary, stabilizing, emulsifying, thickening, coloring agents, flavoring agents, and perfumes may be used.

Compound (I) is included in the pharmaceutical composition in an amount effective to produce the desired effect upon a particular disease process or condition. Preferably, Compound (I) is included in an amount sufficient to provide a neurotropic effect or stimulate nerve cell growth.

Mammals which may be treated using the method of the present invention include livestock mammals such as cows, horses, pigs, etc., domestic animals such as dogs, cats, rats, mice, rabbits, hamsters, etc., primates, and humans.

Preferable modes for the administration or application of products or compositions containing Compound I to humans include injection or oral administration.

While the therapeutically effective amount or dosage of Compound (I) may vary among individual patients and also depends upon the age and condition of each individual patient to be treated, a daily dose ranging from about 0.0001-1000 mg, preferably 0.001-500 mg and more preferably 0.01-100 mg of the active ingredient is generally given for treating diseases, and an average single dose of about 0.001- 0.01 mg, 0.2-0.5 mg, 1 mg, 5 mg, 10 mg, 50 mg, 100 mg, 250 mg and 500 mg is generally administered. Daily doses for chronic administration in humans will be in the range of about 0.1-30 mg/kg/day. Compound (I) may also be administered or applied simultaneously, separately or sequentially with other agents having neurotrophic or nerve cell growth stimulating activity.

Pharmaceutical compositions according to the invention can be periodically administered to a mammalian subject (e.g., a human patient), in need of such treatment, to promote neuronal regeneration and functional recovery and to stimulate neurite outgrowth and thereby to treat various neuropathological states, including damage to peripheral nerves and the central nervous system caused by physical injury (e.g., spinal cord injury and trauma, sciatic or facial nerve lesion or injury, limb transplantation following amputation); disease (e.g., diabetic neuropathy); cancer chemotherapy (e.g., neuropathy induced by acrylamide, taxol, vinca alkaloids and doxorubicin); sequela—e.g. allophasis (such as articulation disorders), clouding of consciousness, dyskinesia, etc. associated with cerebral infarction, hemorrhage infarct, etc.; and neurological disorders including, but not limited to, various peripheral neuropathic and neurological disorders including, but not limited to: trigeminal neuralgia, glosspharyngeal neuralgia, Bell's palsy, myasthenia gravis, muscular dystrophy, amyotrophic lateral sclerosis, progressive muscular atrophy, progressive bulbar inherited muscular atrophy, herniated, ruptured or prolapsed vertebral disk syndromes, cervical spondylosis, plexus disorders, thoracic outlet destruction syndromes, peripheral neuropathles such as those caused by lead, acrylamides, gamma-diketones (glue-sniffer's neuropathy), carbon disulfide, dapsone, ticks, porphyria, Gullain-Barré syndrome, Alzheimer's disease, Parkinson's disease, and Huntington's chorea.

A transsection of a peripheral nerve or a spinal cord injury can be treated by administering a nerve growth stimulating amount of the agent to the mammal and grafting to the peripheral nerve or spinal cord a nerve graft such as an allograft (Osawa et al., J Neurocytol. 19:833-849, 1990; Buttemeyer et al., Ann. Plastic Surgery 35:396-401, 1995) or an artificial nerve graft (Madison and Archibald, Exp. Neurol. 128:266-275, 1994; Wells et al., Exp. Neurol. 146:395-402, 1997). The space between the transected ends of the peripheral nerve or spinal cord is preferably filled with a non-cellular gap-filling material such as collagen, methyl cellulose, etc., or cell suspensions that promote nerve cell growth, such as Schwann cells (Xu et al., J Neurocytol. 26:1-16, 1997), olfactory cells and sheathing cells (Li et al. Science 277:2000-2002, 1997). The nerve growth promoting agent can be included together with such cellular or non-cellular gap-filling materials, or administered systemically before, during or after the nerve graft procedure.

Particularly, compound (I) is useful for treating or preventing the neuronal injury/dysfunction polymyositis (multiple myositis), Guillain-Barré syndrome, Meniere's disease, polyneuritis (multiple neuritis), mononeuritis (solitary neuritis), Alzheimer's disease, Parkinson's disease, amyotrophic lateral sclerosis (ALS), Huntington's disease, radiculopathy, diabetic neuropathy, chemotherapy-induced neuropathy, senile dementia, vascular dementia, multiple sclerosis, physical palsy, or spinal cord injury.

The following examples illustrate the present invention in further detail. It should be understood that these examples describe certain aspects or embodiments of the invention, but are not intended to limit the scope of the invention.

### Example 1

Treatment with Compound (I) Significantly Increases Neurite Lengths of Hippocampal Neurons

Preparation of Cell Cultures:

Embryonic hippocampal neurons were obtained from rat pups on embryonic day 18.5 (“El8.5”), according to Banker and Cowan (Brain Research, 1977, 126: 397425). Briefly, the hippocampal regions were removed, minced, and incubated in 100 I.U. papain at 37° C. for 45 min, and the cells were resuspended in complete neuronal medium: minimal essential medium without L-glutamine (GIBCO, Grand Island, NY), 1.5 ml/100 ml medium of high glucose minimal essential medium (GIBCO), 0.1 ml/100 ml medium of serum extender (Hito +Tm; Collaborative Research Inc, Lexington, Mass.), glutamine (GIBCO), 5% fetal calf serum (GIBCO).

Cells were seeded onto coverslips (500 cells/coverslip) coated with poly-L-lysine. The coverslips were inverted onto dishes that had been precoated with a monolayer of cortical astrocytes.

Analysis of Axonal Lengths in Hippoocampal Neurons:

Hippocampal neurons (identified by their characteristic polarity and dendrites) were examined daily and randomly photographed (9-12 frames/coverslip) at 72 h. Axon (defined as the longest process) lengths were measured on photographic prints using a Houston Instrument HI-PAD digitizing tablet connected to an IBM XT computer with appropriate software (Bioquant IV, R & M Biometrics, Nashville, TN); only processes more than three-fold of the cell body length were measured. Data from identically treated coverslips (three or four per group) were not different and therefore were combined. Mean values were calculated and compared using a one-way (groups treated with Compound (Ia) or tacrolimus versus an untreated control group) ANOVA followed by Newman-Kuels multiple comparisons test (WINKS 4.62 professional edition).

Results:

At 72 hours, there was no significant difference between the untreated control group and the group treated with 10 nM tacrolimus. However, the group treated with a 10 nM concentration of Compound (Ia) elicited a statistically significant increase in length of neurites. See results in Table I below:

TABLE 1
Effects of Compound (Ia) and Tacrolimus on Mean Neurite
Lengths in Primary Hippocampal Cell Cultures in
Rats at 72 hr

| | Neurite Lengths |
|---|---|
| | (μm) |
| No Treatment | 372.3 ± 18.23 |
| Tacrolimus (10 nM) | 423.4 ± 19.50 |
| Compound (Ia) (10 nM) | 502.2 ± 23.61* |

*P < 0.05 versus No Treatment (one-way ANOVA followed by Newman-Kuels multiple comparisons test)

### Example 2

Treatment with Compound (I) Increases Mean Neurite Lengths in SH-SYSY Human Neuroblastoma Cells

Preparation of SH-SY5Y Neuroblastoma Cell Cultures:

SH-SY5Y human neuroblastoma cells were maintained in DMEM medium (GIBCO) supplemented with 10% fetal calf serum (SIGMA), 50 I.U./ml of penicillin, and 50μg/ml streptomycin (GIBCO) at 37° C. in 7% CO₂. Cells were plated in six-well plates at 15,000 cells/well and treated with 0.4 μM aphidicolin (SIGMA). At 5 days, cells were washed and treated with nerve growth factor (NGF) at 10 ng/ml (to induce process outgrowth) in the presence or absence of tacrolimus (10 nM) or Compound (Ia) (1 nM). Medium was changed at 96 h and replaced with fresh medium for an additional 72 h (total time, 168 h). Duplicate wells were run in all experiments and the data were averaged for each treatment group. Analysis of neurite lengths in SH-SY5Y neuroblastoma cells:

For analysis of process length, cells (20 fields per well) were randomly photographed at 168 h. Neurite lengths were measured on photographic prints using a Houston Instrument HI-PAD digitizing tablet connected to an IBM XT computer with appropriate software (Bioquant IV, R & M Biometrics, Nashville, Tenn.); only those processes greater than two-fold of the cell body length were measured. Data from identically treated wells were not different and were therefore combined. Mean values were calculated and compared using a one-way (Compound (Ia) or tacrolimus treated samples versus samples treated with NGF alone) ANOVA followed by Newman-Kuels multiple comparisons test (WINKS 4.62 professional edition).

Results:

Measurement of the lengths of neurite processes demonstrated that both Compound (Ia) (1 nM) and tacrolimus (10 nM) significantly increased the length of neurite processes at 168 h compared to NGF (10 ng/ml) alone. However, the effects of 1 nM Compound (Ia) in combination with NGF were higher than the effects of 10 nM tacrolimus in combination with NGF.

TABLE 2
Effect of Compound (Ia) and Tacrolimus on Mean Neurite
Lengths in SH-SY5Y Human Neuroblastoma Cells at 168 hr

| | Neurite Lengths |
|---|---|
| | (μm) |
| NT | 94.75 ± 3.734 |
| NGF (10 ng/ml) | 198.8 ± 8.991 |
| tacrolimus (10 nM) + NGF (10 ng/ml) | 227.6 ± 9.130* |
| Compound (Ia) (1 nM) + NGF (10 ng/ml) | 256.0 ± 9.067* |

*P < 0.05 versus NGF (one-way ANOVA followed by Newman-Kuels multiple comparisons test)

### Example 3

Treatment with Compound (I) Promotes Functional Recovery in the Rat Sciatic Nerve Crush Model

Animals and Surgical Procedure:

Nine 6-week-old male Sprague-Dawley rats were anesthetized with 2% halothane, the right sciatic nerve was exposed, and the nerve was crushed twice (for a total of 60 s using a No.7 Dumont jeweler's forceps) at the level of the hip. The crush site was marked by tying a sterile 9-0 suture through the epineurial sheath.

Preparation of Compound (Ia) and administration:

Compound (Ia) was dissolved in vehicle comprising 30% dimethylsulfoxide (DMSO):70% saline. Three axotomized rats received subcutaneous daily injections either Compound (Ia) (1 or 5 mg/kg) or an equivalent volume of vehicle (30% DMSO in saline) (5ml/kg)

Behavioral assessment:

Animals were examined daily until the day of perfusion (18 days). The following semi-quantitative scale was used to evaluate the functional recovery of the animals:

0: paralysis with the foot turned-out upon walking and the toes curved;

1: ability to right the foot and move the toes;

2: ability to constantly walk on the foot;

3: demonstrates toe spread during walking;

4: walks off of heel and shows near normal toe spread. Animals demonstrating intermediate abilities were given partial scores: +, 0.25 ; ++, 0.5; +++, 0.75.

Tissue Fixation and Preparation:

At 18 days after nerve crush, the rats were deeply anesthetized with 4% halothane, heparinized, and perfused with 4% paraformaldehyde in 0.1 M sodium phosphate buffer (pH 7.4) for 10s followed by 5% glutaraldehyde (IL) in 0.1 M sodium phosphate buffer (pH 7.4) and fixed at 4° C. for 24 h. Tissues were sampled from the sciatic nerve at a known (5 mm) distance from the crush site. In the present study, only the data from the branch of the posterior tibial nerve supplying the soleus muscle are reported. Tissues were placed in 0.1 M sodium phosphate buffer (pH 7.4), postfixed with 1% osmium tetroxide (in 0.1 M phosphate buffer) for 2.5 h, dehydrated in ethanol and embedded in plastic. Semithin sections were stained with uranyl acetate and lead citrate, mounted on film-supported 75 mesh grids, and examined in a JEOL 100 CX electron microscope.

Morphometric Analysis:

Analysis of axonal calibers was performed in the soleus nerve. The numbers of regenerating myelinated axons were counted using electron microscopy. Mean values and standard errors were calculated for the vehicle-treated group, Compound (Ia) (1 mg/kg)-treated group, and Compound (Ia) (5 mg/kg)-treated group.

Statistical Analysis:

For the behavioral analysis, mean values for recovery of function were compared using one-way ANOVA followed by the Newman-Keuls multiple comparisons test for comparison of individual values. For the morphometric analysis, mean values for the number of axons were compared using one-way ANOVA followed by the Newman Keuls multiple comparisons test for comparison of individual values.

Results:

Functional Recovery:

Functional recovery was observed on days 15-17, and occurred earlier in both 1 mg/kg-treated rats and 5 mg/kg-treated rats than in vehicle-treated rats. See Table 3 below.

TABLE 3
Effect of Compound (Ia) on Functional Recovery of
Sciatic Nerve Injury in Rats

| Functional Score | |
|---|---|
| Vehicle (s.c.) | |
| (30% DMSO in | Compound (Ia) |

| | saline) | 1 mg/kg (s.c.) | 5 mg/kg (s.c.) |
|---|---|---|---|
| Day 15 | 1.67 ± 0.08 | 2.58 ± 0.17* | 3.17 ± 0.08* |
| Day 16 | 1.83 ± 0.08 | 2.83 ± 0.17* | 3.50 ± 0.00* |
| Day 17 | 2.50 ± 0.00 | 3.08 ± 0.22* | 3.50 ± 0.00* |

*P < 0.05 versus Vehicle (one-way ANOVA followed by Newman-Kuels multiple comparisons test)

Electron Microscopy

Morphological examination of the animals was conducted at 18 days following axotomy.

The numbers of regenerating myelinated axons per nerve area (5,000 μm²) were dramatically increased from 5.5±2.7 (mean±SEM) in vehicle-treated rats to 19±2.4 and 20±2.9 in 1 and 5 mg/kg-treated rats, respectively (P<0.05). See Table 4 below.

TABLE 4
Effect of Compound (Ia) on Numbers of Regenerating
Myelinated Axons per Nerve Area (5,000 μm²) in the
Soleus Nerve 18 Days after Sciatic Nerve Crush in Rats

| | Vehicle (s.c.) (30% | Compound (Ia) | |
|---|---|---|---|
| | DMSO in saline) | 1 mg/kg (s.c.) | 5 mg/kg (s.c.) |
| Regenerating | 5.5 ± 2.7 | 19 ± 2.4* | 20 ± 2.9* |
| myelinated | | | |
| axons | | | |

*P < 0.05 versus Vehicle (one-way ANOVA followed by Newman-Kuels multiple comparisons test)

### Example 4

Treatment with Compound (I) Promotes Functional Recovery in the Rat Spinal Cord Injury Model

(1) Methods

Animals and Surgical Procedure

Twenty-eight 6-week-old male Sprague-Dawley rats were anesthetized with 2% halothane, a laminectomy at T10/T11 was performed and a hemisection lesion of spinal cord was performed at the level of T10/T 11 spinal cord.

Preparation of Compound (Ia) and administration

Compound (Ia) was dissolved in vehicle comprising 30% dimethylsulfoxide (DMSO): 70% saline. The spinal cord lesioned rats received subcutaneous daily injections the Compound (Ia) (2 mg/kg) or an equivalent volume of vehicle (30% DMSO in saline) (5ml/kg) for seven weeks following the surgery.

Evaluation of Functional Recovery

Functional recovery was assessed using a modified Tarlov/Klinger scale, narrow beam test and footprint test at 2 weeks postlesion.

A. modified Tarlov/Klinger scale

Rats were allowed to move freely in an open field for 1 min and rated 0-6 according to the scale presented below.

0: No movement of the lesioned hind limb

1: Barely perceptible movement in the lesioned hind limb

2: Brisk movements at the lesioned hind limb joints (Hip, knee or ankle) but no coordination, no weight support

3: Alternate stepping and propulsive movements of the lesioned hind limb, no weight support

4: Can support weight on the injured hind limb

5: Walk with only mild deficit

6: Normal walking

B. Narrow Beam Test

Rats were tested on wooden beams (1.5 m long) with decreasing width: 7.7 cm, 4.7 cm, 2.7 cm and 1.7 cm. Rats were allowed to walk on the bars, and the narrowest bar they could walk on without any slips in at least two trails was recorded.

0: No walking on any beam

1: Can walk on the 7.7 cm beam

2: Can walk on the 4.7 cm beam

3: Can walk on the 2.7 cm beam

4: Can walk on the 1.7 cm beam

C. Footprint Test

The hind limbs of rats were inked and footprint were made on paper covering a narrow runway of 60 cm length and 7.5 cm width. A series of at least six sequential steps was used to determine the 5-point footprint score.

0: Constant dorsal stepping or hind limb dragging, i.e. no footprint is visible

1: Has visible toe prints of at least three toes in at least three footprints

2: Shows exo-or endo-rotation of the feet of more than double values as compared to its own baseline values

3: Shows no signs of toe dragging but foot rotation

4: Shows no signs of exo-or endo-rotation (less than twice the angle of the baseline values), but more than one heel print are visible

5: No heel prints are visible

Statistical Analysis

For the behavioral analysis, mean values for score of each functional test were compared using one-way ANOVA followed lay the Newman-Keuls multiple comparisons test for comparison of individual values.

(2) Results

Functional Recovery

In all three functional recovery measurements carried out using a modified Tarlov/Klinger scale (Table 5), Beam walking test (Table 6) and footprint test (Table 7) Compound (Ia) improved motor functional impairment in modified Tarlov/Klinger scale (Table 5), Beam walking test (Table 6) and footprint test (Table 7).

TABLE 5
Effect of Compound (Ia) on modified Tarlov/Klinger score
of spinal cord injury in rats

Modified Tarlov/Klinger score

| | Vehicle (s.c.) | Compound (Ia) |
|---|---|---|
| | (30% DMSO in saline) | 2 mg/kg (s.c.) |
| Week 2 | 2.1 ± 0.1 | 3.7 ± 0.2* |

*P < 0.05 versus Vehicle (one-way ANOVA followed by Newman-Kuels multiple comparisons test)

TABLE 6
Effect of Compound (Ia) on beam walking score of spinal
cord injury in rats

Beam walking score

| | Vehicle (s.c.) | Compound (Ia) |
|---|---|---|
| | (30% DMSO in saline) | 2 mg/kg (s.c.) |
| Week 2 | 0.9 ± 0.1 | 2.0 ± 0.3* |

*P < 0.05 versus Vehicle (one-way ANOVA followed by Newman-Kuels multiple comparisons test)

TABLE 7
Effect of Compound (Ia) on footprint score of spinal
cord injury in rats

Footprint score

| | Vehicle (s.c.) | Compound (Ia) |
|---|---|---|
| | (30% DMSO in saline) | 2 mg/kg (s.c.) |
| Week 2 | 1.7 ± 0.4 | 3.6 ± 0.3* |

*P < 0.05 versus Vehicle (one-way ANOVA followed by Newman-Kuels multiple comparisons test)

### Example 5

Compound (Ia) Binds to FKBP12, but Unlike Tacrolimus Exerts Little or No Immunosuppressive Effect

(1) Binding Assay to FKBP12

The binding assay was performed according to a similar manner to that of Tamura, K., et al (Biochemical arid Biophysical Research Communications, Vol. 202, No. 1, 437-499, 1994). The results are shown in Table 8.

(2) Mixed lymphocyte reaction (MLR)

MLR test was performed according to a similar manner to that of U.S. Pat. No. 4,929,611

The Results are shown in the Table 8.

TABLE 8
Pharmacological profiles of Compound (Ia) and tacrolimus
in vitro

| | FKBP12 binding IC₅₀ | MLR IC₅₀ |
|---|---|---|
| | (nM) | (nM) |
| Compound (Ia) | <5 | >100 |
| Tacrolimus | <5 | <2 |

The above results indicate that the compound (Ia) does not have immunosuppressive activity though it can bind to FKBP12.

The results shown above illustrate the potent neurotrophic effects of Compound (I) using both in vitro and in vivo models. In two cell culture models, Compound (I) even at low concentrations increased neurite outgrowth. Moreover, systemic administration of Compound (I) at low doses speeded functional recovery following a nerve crush lesion by increasing the rate of axonal regeneration in the sciatic nerve and promoted functional recovery from spinal cord injury.

Moreover, as shown above, Compound (I) provides a potent neurotrophic or nerve cell growth stimulating activity, though it has no immunosuppressive activity. Accordingly, the present invention provides a useful neurotrophic agent for stimulating or promoting neuronal growth or regeneration, particularly when an immunosuppressive effect is not advantageous or desired.

Other aspects of the present invention include:

An article of manufacture, comprising packaging material and Compound (I) identified in the above contained within said packaging material, wherein said Compound (I) is therapeutically effective for preventing or treating neuronal dysfunction, and wherein said packaging material comprises a label or a written material which indicates that Compound (I) can or should be used for preventing or treating neuronal injury/dysfunction.

A commercial package comprising the pharmaceutical composition containing Compound (I) identified in the above and a written matter associated therewith, wherein the written matter states that Compound (I) can or should be used for preventing or treating neuronal injury/dysfunction.

A composition, such as a cell suspension, tissue, or graft comprising a cell treated with Compound (I). Such compositions are useful for repairing damage to the nervous system. Such compositions may also include other nerve cell growth stimulating agents, such as other types of cell suspensions that promote or assist nerve cell growth, such as myelin-producing cells such as Schwann cells or oligodendrocytes, glial cells and sheathing cells; extracellular matrix material, such as collagen; or other specific neuroregulators such as cytokines, mitogenic factors, immunophilins, and neurotrophins, such as NGF-1, BDNF, CNTF, NT-3, NT-4 and NT-5.

Grafts, such as homografts, allografts or xenografts may also be treated with Compound (I) in order to facilitate neuronal outgrowth and their use as transplants and for other applications.

Incorporation by Reference

The content of each document, patent application or patent publication cited by or referred to in this disclosure is incorporated by reference in its entirety. The content of any patent document to which this application claims priority is also incorporated by reference in its entirety. Specifically, the content of U.S. Provisional Application No. 60/258,500 is incorporated by reference.

Modifications and other embodiments

Obviously, numerous modifications and variations of the present invention are possible in light of the above teachings. It is therefore to be understood that within the scope of the appended claims, the invention may be practiced otherwise than as specifically described herein. Various modifications and variations of the described compositions and methods, as well as the concept of the invention, will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, biological, chemical or pharmacological arts or related fields are intended to be within the scope of the present invention.

1. A use of a compound of the following formula: 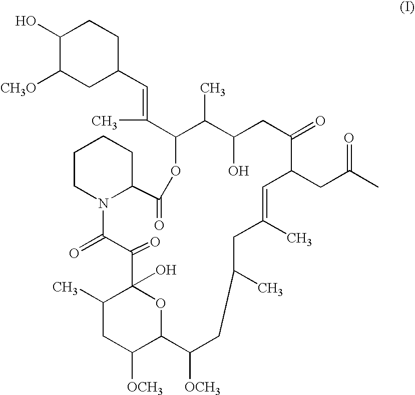
for manufacturing a neurotrophic agent.

2. The use in claim 1, in which the neurotrophic agent is for preventing or treating neuronal injury/dysfunction.

3. The use of claim 1, wherein the neurotropic agent is for stimulating or promoting nerve cell growth or regeneration.

4. The use of claim 1, wherein the neurotropic agent is for promoting functional recovery from a nerve injury.

5. The use of claim 1, in which the neuronal injury/dysfunction is polymyositis (multiple myositis), Guillan-Barre syndrome, Meniere's disease, polyneuritis (multiple neuritis), mononeuritis (solitary neuritis), Alzheimer's disease, Parkinson's disease, amyotrophic lateral sclerosis (ALS), Huntington's disease, radiculopathy, diabetic neuropathy, chemotherapy-induced neuropathy, senile dementia, vascular dementia, multiple sclerosis, physical palsy, or spinal cord injury.

6. The use of claim 1, in which the neuronal injury/dysfunction is Alzheimer's disease, Parkinson's disease, amyotrophic lateral sclerosis, diabetic neuropathy, chemotherapy-induced neuropathy, or spinal cord injury.

7. The use of claim 1, in which the compound (I) is in a form of its pharmaceutically acceptable salt, derivative, pro-drug or solvate.

8. A composition comprising Compound (I) having the following formula:

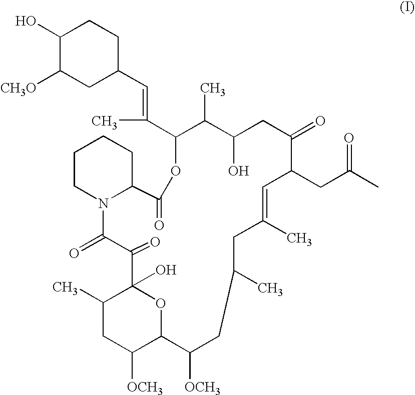

9. The composition of claim 8, wherein Compound (I) is in a form of its pharmaceutically acceptable salt, derivative, pro-drug or solvate.

10. The composition of claim 8, wherein Compound (I) is in the form of Compound (Ia).

11. The composition of claim 8, further comprising at least one other neurotrophic agent.

12. An article of manufacture or a kit, comprising:
packaging material and
Compound (I) contained within said packaging material,
wherein said packaging material comprises a label or a written material which indicates that said Compound (I) can or should be used for preventing, ameliorating or treating neuronal injury/dysfunction.

13. A commercial package or kit comprising:
a pharmaceutical composition comprising Compound (I) and
written matter associated therewith,
wherein the written matter states that Compound (I) can or should be used for preventing, ameliorating or treating neuronal injury/dysfunction.

14. A method for making or manufacturing a neurotrophic agent comprising mixing Compound (I) with a pharmaceutically acceptable carrier, vehicle or excipient.

15. A method of preventing, ameliorating or treating neuronal injury/dysfunction comprising administering an effective amount of Compound (I) to a subject in need thereof.

16. The method of claim 15, wherein said subject is a mammal.

17. The method of claim 15, wherein said subject is a human.

18. The method of claim 15, wherein said neuronal injury/dysfunction is polymyositis (multiple myositis), Guillan-Barre syndrome, Meniere's disease, polyneuritis (multiple neuritis), mononeuritis (solitary neuritis), Alzheimer's disease, Parkinson's disease, amyotrophic lateral sclerosis (ALS), Huntington's disease, radiculopathy, diabetic neuropathy, chemotherapy-induced neuropathy, senile dementia, vascular dementia, multiple sclerosis, physical palsy, or spinal cord injury.

19. The method of claim 15, in which the neuronal injury/dysfunction is Alzheimer's disease, Parkinson's disease, amyotropic lateral sclerosis, diabetic neuropathy, chemotherapy-induced neuropathy, or spinal cord injury.

20. A method for stimulating or promoting nerve cell growth or regeneration comprising contacting a nerve cell with Compound (I).

21. A method for increasing the rate of axonal growth or regeneration, or nerve cell length, comprising contacting a nerve cell with Compound (I).

22. A method of increasing nerve cell growth in a tissue in need thereof, comprising administering Compound (I) to said tissue.

23. The method of claim 22, wherein said tissue is selected from the group consisting of brain tissue, spinal cord tissue or peripheral nerve tissue.

24. A method of promoting functional recovery from a nerve injury comprising administering an effective amount of Compound (I) to a subject in need thereof.

25. The method of claim 24, wherein said nerve injury is selected from the group consisting of a burn, traumatic injury, mechanical injury, surgical injury, physiological injury, pathological injury and immunological injury.

26. A method for repairing a transected peripheral nerve or spinal cord comprising contacting the transected ends of said peripheral nerve or spinal cord with an effective amount of Compound (I).

27. A method for repairing a transected peripheral nerve or spinal cord in a subject comprising:
administering a nerve growth stimulating amount of Compound (I) to said subject, and
grafting to the peripheral nerve or spinal cord.

28. The method of claim 27, wherein said graft is an allograft.

29. The method of claim 27, wherein said graft is an artificial nerve graft.

30. The method of claim 27, further comprising filling the space between the transected ends of the peripheral nerve or spinal cord with a noncellular gap-filling material.

31. The method of claim 27, further comprising filling the space between the transected ends of the peripheral nerve or spinal cord with a cell suspension.

32. A composition comprising a nerve cell treated with Compound (I).

33. A tissue comprising a nerve cell treated with Compound (I).

34. A graft comprising a nerve cell treated with Compound (I).

35. The graft of claim 34, wherein said graft is a homograft, an allograft or a xenograft.

36. A method of promoting functional recovery from a nerve injury comprising administering an effective amount of a composition comprising a cell treated with Compound (I) to a subject in need thereof.

37. The method of claim 36, wherein said nerve injury is selected from the group consisting of traumatic injury, mechanical injury, surgical injury, physiological injury, pathological injury and immunological injury.

38. A method for repairing a transected peripheral nerve or spinal cord comprising contacting the transected ends of said peripheral nerve or spinal cord with an effective amount of a composition comprising a cell treated with Compound (I).

39. A composition comprising a cell, tissue or graft treated with Compound (I) and at least one nerve cell growth promoting agent.

40. The composition of claim 39, wherein said nerve growth promoting agent is selected from the group consisting of a cell suspension that promotes neural growth, a noncellular gap-filling material, or a nerve growth factor.

41. The composition of claim 39, comprising collagen or methyl cellulose as a nerve growth promoting agent.

42. The composition of claim 39, comprising a cell suspension as a nerve growth promoting agent.
